(19) United States
(12) Patent Application Publication
(10) Pub. No.: US 2014/0088185 A1
(43) Pub. Date: Mar. 27, 2014

(54) METHOD OF TREATING DIGESTIVE UPSETS BY BUCCAL ADMINISTRATION OF BOTANICAL DERIVATIVES

(71) Applicant: Enteral Health and Nutrition, Chesterfield, MO (US)

(72) Inventors: Dustin Garth James, St. Louis, MO (US); Helen Kim-James, St Louis, MO (US)

(21) Appl. No.: 14/091,877

(22) Filed: Nov. 27, 2013

Related U.S. Application Data
(63) Continuation-in-part of application No. 13/328,114, filed on Dec. 16, 2011.

(51) Int. Cl. A61K 9/00; A61K 31/12; A61K 31/11; A61K 31/085; A61K 31/22; A61K 31/045; A61K 31/216; A61K 31/122; A61K 31/35; A61K 31/05; A61K 31/015

(52) U.S. Cl. CPC A61K 9/0056; A61K 31/05; A61K 31/12; A61K 31/11; A61K 31/085; A61K 31/015; A61K 31/045; A61K 31/216; A61K 31/122; A61K 31/35; A61K 31/22
USPC 514/456; 514/731; 514/678; 514/701; 514/717; 514/763; 514/679; 514/739; 514/546; 514/688; 514/681

(57) ABSTRACT

A buccally administered dosage for assisting with the relief of common digestive complaints where the dosage formed into a hard candy dosage drop, where the dosage is a sugar base infused with a botanical derivative that has concentrated active compositions in sufficient concentration to provide a soothing effect of digestive upset, and where the drop has favorable organoleptic properties.

### CROSS-REFERENCE TO RELATED APPLICATIONS

The present application claims priority to U.S. Provisional Application Ser. No. 61/490,203 filed May 26, 2011 and having the title “METHOD OF ASSISTING WITH DIGESTIVE UPSETS USING A CONFECTION-BASED DELIVERY OF PEPPERMINT OIL AND GINGER”, which is hereby incorporated by reference, and utility patent application Ser. No. 13/328,114 having the same title, filed Dec. 16, 2011, which is hereby incorporated by reference.

### FIELD OF THE INVENTION

This invention relates to the treatment of digestive upsets by the buccal administration of botanical derivatives.

### BACKGROUND OF THE INVENTION

Botanical derivatives, including teas, powders, extracts and essential oils have been used for treating digestive upsets. The challenge in using the botanical derivatives is consuming enough of the active ingredient to provide the desired digestive relief. Many botanical derivatives which have concentrated active compounds, such as essential oils and extracts are harsh and irritating. When doses are sufficiently large to be effective, the organoleptic properties are too unpleasant and/or irritating.

There has been an approach of taste-masking the botanical derivatives to deliver them in sufficient quantity to be effective. One such approach is to encapsulate the active. Heather's Tummy tamers contain essential oils in a gelatin capsule. However, when a capsule is used, the delivery of the active is not immediate, as the capsule needs to be ingested and then released into the stomach and small intestine for absorption. It is desirable to have an immediate release dosage. Additionally, it is desirable to have the botanical active absorbed before reaching the stomach and small intestine so that the digestive system does not begin to break down the active. In addition, the rapid absorption of the botanical active from the buccal route is desirable in that it bypasses first-pass hepatic metabolism. Active botanicals absorbed from the stomach and small intestine subject to degradation by the liver before they enter the systemic circulation. In the buccal route, the rich vascular supply of the buccal mucosa and salivary glands directly feeds into the jugular venous system which directly drains into the heart for systemic circulation.

Hard candies have been made with essential oils which flavor the candy, but not enough of the essential oils are added to the candy to soothe digestive upsets, because essential oils can be harsh and/or irritating when concentrated. An embodiment of the present invention includes at least one additional natural flavoring with the essential oil in the hard candy drop which provides an improved hard candy that is both soothing to digestive discomforts and has pleasant organoleptic properties.

The present buccal delivery system provides the required dose of botanical active in an organoleptically pleasing form that is immediately released. While some of the derivative may be swallowed as it dissolves in the mouth, there is absorption through the membranes of the mouth, throat, and salivary glands, thereby by-passing the degradation and latency of the luminal digestive system.

The present buccal delivery system provides unencapsulated botanical derivatives in an immediate release form that are in a sufficiently high dosage to be effective in relieving digestive distress. The dosage form is a hard candy delivery system, and the botanical extracts are added either neat or highly concentrated during the process of heating the sugar base to prepare the candy. Optionally, other natural flavorings are added to make the dose palatable. These range from 0.002% to 1% by weight.

### SUMMARY OF THE INVENTION

The present invention is a solid buccal delivery system for treating digestive distress comprising a hard candy dosage form infused with botanical derivatives, wherein the botanical derivatives have concentrated active compounds; wherein the active compounds are present in an amount sufficient to provide relief of the digestive distress, where the botanical derivatives are not encapsulated, and wherein the active compounds are immediately released in the oral cavity. Immediate release is understood in the art to be at least partially released in about 15 minutes or less.

The present invention further comprises a method of preparing a buccal delivery system for treating digestive distress comprising heating a sugar base, adding the botanical derivative neat or concentrated form to the base, pouring the base into the desired dosage shapes, and cooling the base to form a solid dosage, wherein the botanical derivatives are present in an amount sufficient to provide relief of the digestive distress, where the botanical derivatives are not encapsulated, and where the botanical derivatives are immediately released.

The presently disclosed subject matter will be better understood from reading the following description of non-limiting embodiments, with reference to the attached drawings, wherein below:

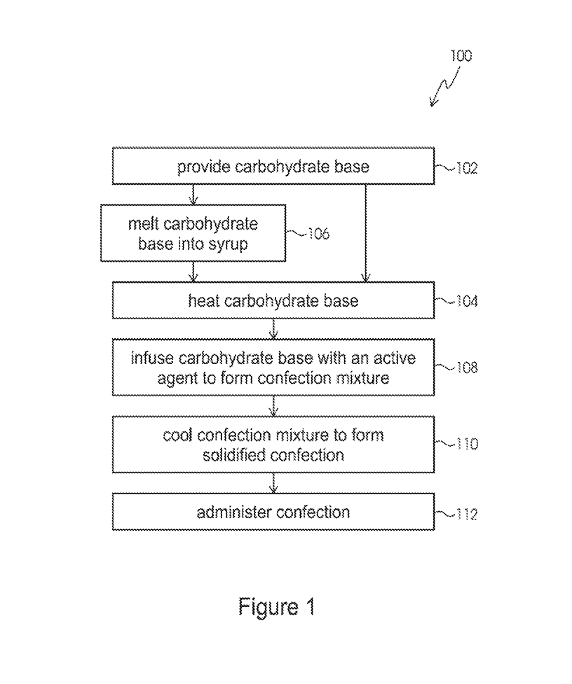

FIG. 1 is a flowchart of a method for forming the hard candy dosage for digestive disorders

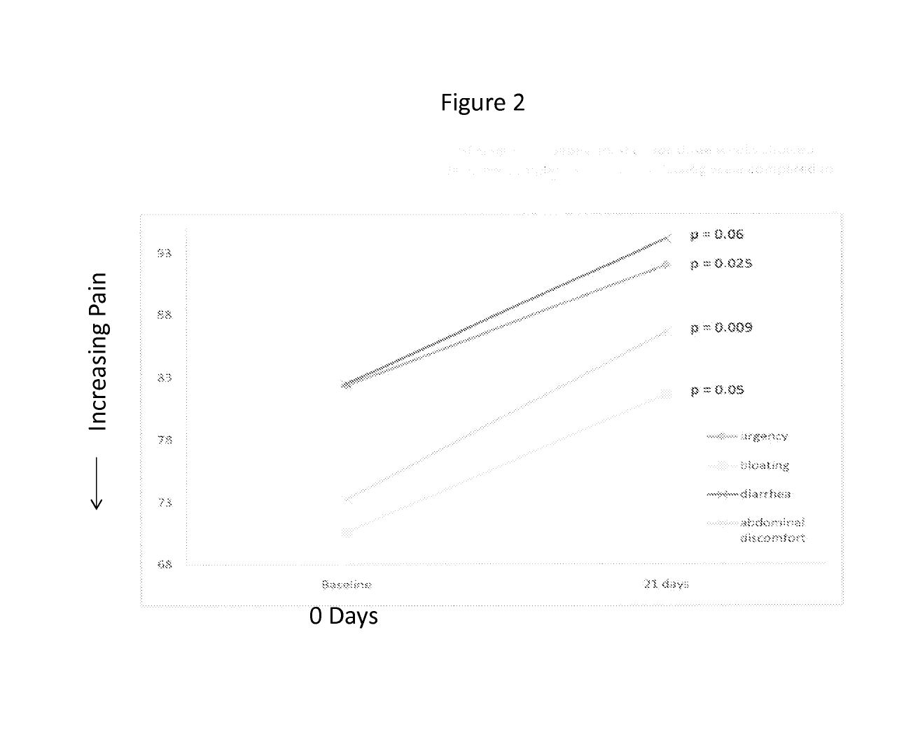

FIG. 2 is an x-y graph showing the results of clinical trials.

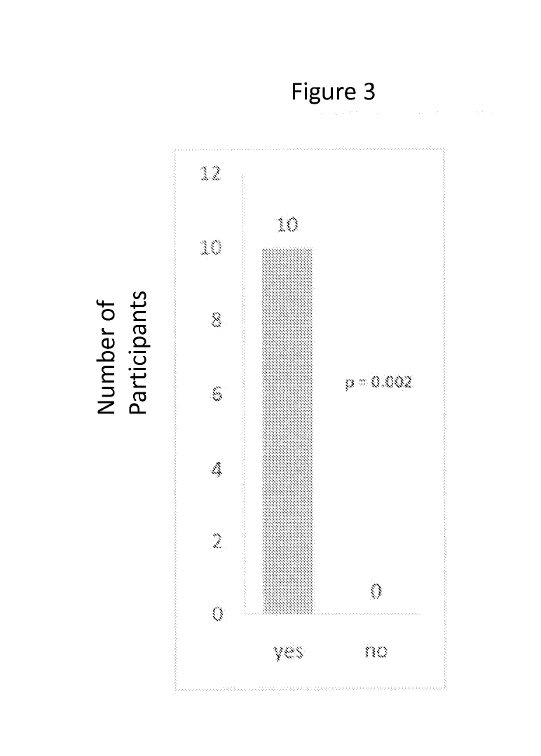

FIG. 3 is a bar graph showing immediate improvements in common digestive symptoms after treatment with the present invention.

Various embodiments provide a hard candy for assisting with the relief of common digestive complaints, wherein the sugar base contains a sufficient amount of botanical derivative. A list of suitable botanical derivatives is given in Table 1 below. These botanicals are recognized as GRAS, which stands for Generally Recognized as Safe.

TABLE 1

| | Essential | | | |
|---|---|---|---|---|
| | oil % of | Extract | Digestive and | Major |
| Botanical | dry wt. | Taste | Health Effects | chemicals |
| Lavender | 3 | Extremely | Antispasmodic, bowel | Linalool, Linalyl |
| Lavandula | | bitter | urgency, nervous | acetate, Cineole |
| officinalis | | | stomach, relaxation | |
| | | | promoting, dyspepsia, | |
| | | | sleep promoting | |
| Rosemary | 2.5 | Extremely | Dyspepsia, | 1,8-cineole, alpha |
| Rosmarinus | | bitter | antispasmodic, | pinene, camphor |
| officinalis | | | promotes alertness and | |
| | | | cognitive performance | |
| Cinnamon | 1 | Injurious to | Blood sugar control, | Cinnamic aldehyde, |
| Cinnamomum | | mucosa, | dyspepsia, bloating | cinnamyl acetate, o- |
| species (bark) | | intense | | methoxy |
| | | pungency | | cinnamaldehyde |
| Fennel | 12 | Cloying | Carminative, bloating, | Anethole, fenchone |
| Foeniculum | | sweetness, | infantile colic, | |
| vulgare (fruit) | | negative | dysmenorrhea, | |
| | | organoleptic | intestinal spasms | |
| Turmeric | Nonvolatile: | Bitter | Dyspepsia, | Curcuminoids- |
| Curcuma | curcuminoids | | inflammation, irritable | nonvolative |
| longa | 1.5 | | bowel syndrome | derivative |
| | Volatile: | | | Turmerone-volative |
| | turmerones: 3 | | | derivatives |
| Caraway | 3 | Bitter, very | Spasms, dyspepsia | Carvone, Limonene |
| Carum carvi | | unpleasant | | |
| | | organoleptics | | |
| Peppermint | 1 | Injurious to | Irritable Bowel | Menthol, Menthone, |
| Mentha x | | mucosa, | Syndrome, intestinal | cineole |
| piperita | | bitterness, | cramps and urgency, | |
| | | intense | dyspepsia, nausea, | |
| | | menthol | alertness, diarrhea, | |
| | | organoleptic | abdominal pain | |
| Ginger | 1.8% | Intense | Pregnancy nausea and | Gingerol |
| Zingiber | nonvolatile | pungency, and | vomiting, dyspepsia, | |
| officinale | gingerols | potentially | motion sickness, | |
| | | injurious to | vertigo, nausea, | |
| | | mucosa | nausea and vomiting | |
| | | | related to cancer | |
| | | | treatment | |

Fresh botanicals contain about 95% water. Of the remaining mass, biologically active compounds only represent a small percentage. It is desirable to administer an effective amount of active compounds. In fresh form, however, this would require consuming an excessive amount of botanical, as active compounds are typically less than 0.15% by weight per fresh botanical.

Dried botanicals such as culinary spices contain from 0.16 wt. % to 7 wt. % by weight active beneficial compounds, with a typical range from 0.5% to 2.5% by weight. Dried botanicals would require an excessive amount to be consumed in order to ingest the desired amount of active compounds.

Botanical extracts can be in the form of essential oil, powder, or a liquid are processed in some way to enrich the active compounds. These processes include steam distillation, supercritical CO₂ extraction, solvent extraction, or other methods. The active compounds are present in these extracts from about 10 wt. % to 100 wt. %. Since the active compounds are concentrated, small quantities are used to ingest the desired amount of active. However, prior art dosages have encapsulated concentrates, because of the strong taste and irritating character of these concentrates. A table of such botanical extracts with typical amounts of active compounds is given below.

The amount of active compound present in the hard candy drops can vary from about 0.5 mg to 50 mg. The amount of active compound is present from about 0.01 to about 5.0 wt. %. Preferably, the amount of active compound present in the hard candy drop is in the range from about 1.0 mg to 40 mg. The drops weigh from about 1.0 to 5.4 g total, and preferably, from about 3.0 to 5.0 g. Lower dosages fail to provide soothing properties for stomach upset, and higher dosages are often too irritating. The addition of additional flavorings provides improved palatability that allows the clinically meaningful doses of active compounds to be achieved.

TABLE 2
Concentrated Botanical Derivatives

| Botanical Derivative | Wt. % Active |
|---|---|
| Ginger (dry extract) | |
| Gingerol | 10 |
| Peppermint (essential oil) | |
| Menthol | 44 |
| Menthone | 21.5 |
| Cineole | 4.3 |
| Fennel (essential oil) | |
| Anethole | 69.96 |
| Fenchone | 15.01 |
| Cinnamon bark (essential oil) | |
| Cinnamic Aldehyde | 79.48 |
| O-methoxy cinnamaldehyde | 8.92 |
| Cinnamyl Acetate | 3.29 |
| Lavender (essential oil) | |
| Linalool | 34.07 |
| Linalyl acetate | 27.12 |
| Rosemary (essential oil) | |
| Cineole | 43.30 |
| Camphor | 11.83 |
| Alpha Pinene | 11.09 |
| Turmeric | |
| Nonvolatiles | |
| Curcuminoids | 95 |
| Volatiles | |
| Turmerones | 88 |
| Caraway (essential oil) | |
| D-Carvone | 54.81 |
| D-Limonene | 41.36 |

In a preferred embodiment, the botanical derivative is combined with a natural flavoring, where the concentration of the flavoring is less than or equal to 1.0 wt. %, which, unexpectedly, provides improved palatability. For some botanical derivatives, citric acid, in the range of 0.1 to 0.5 wt. % also improves palatability. Table 3, below summarizes some of the combinations.

TABLE 3

| | Flavoring - | | |
|---|---|---|---|
| Botanical | Improved | Flavoring - No | |
| Derivative | palatability | improvement | Citric Acid |
| Ginger | Strawberry, | Cinnamon. | No |
| | raspberry | Pungency enhanced | |
| | | with deleterious | |
| | | consequences on | |
| | | organoleptics. | |
| Peppermint | Vegan cream | | No |
| Fennel | Blueberry, orange | | Yes |
| Lavender | Orange | Vanilla | Yes |
| Caraway | Apple | Cherry | Yes |
| Tumeric | Mango and coconut | Orange | |
| | combination | | |
| Rosemary | Lemon | | Yes |
| Cinnamon | Cranberry | Cinnamon roll. | Yes |
| | | Additional | |
| | | pungency had | |
| | | deleterious | |
| | | consequences on | |
| | | organoleptics. | |

### EXAMPLES OF THE INVENTION

The botanical derivative may be infused into the sugar base in a range of 0.1% to 5.0% by weight. The amount of active compound is present from about 0.01 to about 5.0 wt. %. Table 4 provides an embodiment of a typical sugar base suitable for the present invention.

TABLE 4
Base Formula

| Ingredients | Percentage |
|---|---|
| Brown rice syrup | 45-55% |
| Dried cane syrup | 45-55% |
| Citric acid | 0-1% |
| Natural flavors | < or = 1% |
| Active ingredients | Listed below |

### Controls 1-6 and Examples 1 and 2

Method of Preparation of Botanical Derivative Drops

The sugar base described in Table 4 is heated and melted to form a liquid. The liquid is held at 242 degrees F. for about 15 minutes at which time the water in the syrup is lowered to between about 1.5 wt. % to 3 wt. %. For Controls 1-4 and Example 1, ginger derivative is infused into the liquid base in the form of ginger extract powder. This powder contains a concentrated 10% gingerol content prepared by the CO₂ extraction method. The liquid is cooled to form the confection drops.

For Controls 5 and 6, and Example 2, Peppermint Oil is blended into the above-described liquid base in the form of peppermint oil. The optimal peppermint oil has typical concentrations of 44% menthol, 21.5% menthone and 4.3% cineole. The liquid is cooled to form the confection drops. A panel of 5 adults evaluated the following drops, as summarized in Table 5.

TABLE 5

| | | | | Dose of | |
|---|---|---|---|---|---|
| | | | | Major | |
| | | | | Active | |
| | | | | Botanical | |
| | Botanical | Major | Total wt. of | Derivatives | |
| Sample | Derivative | Active Wt. % | drop (g) | (mg) | Comments |
| Control 1 | Ginger | 10% gingerol | 5.4 | 4.9 | Drop too |
| | Extract | | | | large leading |
| | Powder | | | | to cumulative |
| | | | | | negative |
| | | | | | organoleptics |
| Control 2 | Ginger | 10% gingerol | 3.6 | 5.0 | Ideal piece |
| | Extract | | | | size, too |
| | Powder | | | | pungent to |
| | | | | | tolerate |
| Control 3 | Ginger | 10% gingerol | 3.6 | 2.5 | Too pungent |
| | Extract | | | | |
| | Powder | | | | |
| Example 1 | Ginger | 10% gingerol | 3.6 | 1.5 | Soothing and |
| | Extract | | | | acceptable |
| | Powder | | | | |
| Control 4 | Ginger | 10% gingerol | 3.6 | 1.0 | Not soothing |
| | Extract | | | | |
| | Powder | | | | |
| Control 5 | Peppermint | 44% menthol, | 5.4 | 70 | Piece too |
| | Oil | 21.5% | | | large, cooling |
| | | menthone, | | | effect of |
| | | and 4.3% | | | menthol was |
| | | cineole | | | too intense |
| Control 6 | Peppermint | 44% menthol, | 3.6 | 25 | Less |
| | Oil | 21.5% | | | soothing, |
| | | menthone, | | | piece size |
| | | and 4.3% | | | ideal |
| | | cineole | | | |
| Example 2 | Peppermint | 44% menthol, | 3.6 | 34 | Soothing and |
| | Oil | 21.5% | | | acceptable |
| | | menthone, | | | organoleptic |
| | | and 4.3% | | | properties. |
| | | cineole | | | |

### Examples 3-7 and Controls 7-15

Examples 3-7 and Controls 7-15 were prepared as described above for Example 1 in 3.6 g drops. The samples were evaluated by 5 people for organoleptic properties and by 5 people for stomach soothing properties. A rating of 5 indicates that 5 out of 5 people rated the sample as good. The results are shown in Table 6, below.

TABLE 6

| | | | Dose of | | | | |
|---|---|---|---|---|---|---|---|
| | | | Major Active | | | Good | |
| | | | Botanical | | | Organo- | |
| | Essential | Amount | Derivatives | Flavor | | leptic | Soothing |
| Sample | Oil | (mg) | (mg) | Added | Comments | Properties | Properties |
| Example 3 | Cinnamon 1 | 35 | 32.0 | Cranberry | Most | 5-yes | 5-yes |
| | | | | | tolerable | | |
| Control 7 | Cinnamon1 | 40 | 37.0 | Cranberry | Too | | |
| | | | | | pungent. | | |
| Example 4 | Fennel | 10 | 8.5 | Blueberry | Best | 5-yes | 5-yes |
| Control 8 | Fennel | 15 | 12.8 | Blueberry | Cloyingly | | |
| | | | | | sweet | | |
| Control 9 | Fennel | 22.5 | 19.1 | Blueberry | Cloyingly | | |
| | | | | | sweet | | |
| Example 5 | Caraway | 7.5 | 7.2 | Apple | Best | 5-yes | |
| | | | | | organo- | | |
| | | | | | leptics | | |
| Control 10 | Caraway | 15 | 14.4 | Apple | Too intense | | 5-yes |
| Example 6 | Tumeric2 | 20 | | Mango and | Best | 5-yes | 5-yes |
| | | | | Coconut | | | |
| Control 11 | Tumeric3 | 50 | | Mango and | Harsh and | | |
| | | | | Coconut | bitter | | |
| Control 12 | Tumeric4 | 30 | | Mango and | Very bitter | | |
| | | | | Coconut | | | |
| Control 13 | Rosemary | 15 | 13.0 | Lemon | | 2-yes | |
| Control 14 | Rosemary | 20 | 17.1 | Lemon | | 3-yes | |
| Example 7 | Lavender | 10 | 6.1 | Blood | | 5-yes | 4-yes |
| | | | | orange | | | |
| Control 15 | Lavender | 15 | 9.2 | Blood | Too bitter. | | 1-yes |
| | | | | orange | | | |

Clinical Data

Design

Subjects who experienced at least one digestive complaint in the 3 weeks leading up to enrollment were recruited for a three week open-label study of Example 1 with the present invention of ginger derivative extract with 10% gingerol and Example 2 with peppermint oil to assist with the above digestive complaints.

Subjects were instructed to take the hard candy drops when they experienced digestive upsets. If the subjects did not experience symptoms on a daily basis, they were asked to take the inventions on a daily basis regardless. Subjects were asked record their responses in two forms. One was with a visual analog scale (VAS) where they would mark on a scale of zero (worst) to 100 (best) how they felt overall during the week of study. For subjects who experienced digestive upsets during the study period, they were also asked if they had rapid improvement of their digestive complaints after taking the invention in a simple yes/no answer.

The data supported a statistically significant improvement in digestive symptoms in subjects taking the invention.

FIG. 1 is a flowchart of a method 100 for forming a hard candy for treating digestive disorders. The hard candy releases active compounds from a botanical derivative rapidly into the body through buccal delivery to thereby provide swift relief of common digestive upsets including cramping, urgency, stomach upset, nausea, and bloating. At step 102, a sugar base is provided. The sugar base may be provided in a syrup form. Optionally, the sugar base may be provided in a solid or crystallized form that is melted. In one embodiment, the sugar base includes at least one of sugar from a source including at least one cane syrup, brown rice syrup, or corn syrup, or a sugar-free base including isomalt. Optionally, the sugar base may be a sugar alcohol base, such as isomalt.

The sugar base may be heated to an acceptable temperature for being infused with active agents, at step 104. For example, the sugar base may be heated to as much as 325 degrees Fahrenheit. In an embodiment where the sugar base is provided in a syrup form, the syrup may be heated directly. Alternatively, the syrup may not be required to be heated to infuse the active agent. In an embodiment where the sugar base is provided in a solid or crystallized form, the sugar base may first be melted, at step 106, to form syrup or a heated liquid. The syrup or heated liquid may then be heated for infusion, at step 104.

At step 108, the sugar base is infused with a botanical derivative containing an active agent. In one embodiment, the active agent is gingerol. Optionally, the active agent includes a combination of menthol, menthone and cineole from peppermint oil. The active agent is infused into the sugar base in a concentration that allows for an acceptable organoleptic profile and efficacy for relief of above stated digestive symptoms. The concentration of the botanical derivative ranges from about 0.01 to 5.0% by weight. The amount of active per drop (dose) is from about 1 to 50 mg.

The sugar base is infused with the active agent after the heat is removed from the carbohydrate base, but while the temperature is still high enough for mixing the carbohydrate base and the active agent. Optionally at least one additional natural flavoring may be infused with the botanical derivative. The sugar base and the active agent are mixed to form a substantially uniform confection mixture. At step 110, the sugar mixture is cooled to form the hard candy drop. The hard candy base by the standard method for those skilled in the art of hard candy confectionary making. When ingested, the confection is configured to dissolve into saliva through normal human physiologic prehension process. Although some active botanical derivatives are swallowed, the rich vascular supply of the buccal cavity provide for the rapid absorption of active compounds into systemic circulation. The hard candy maintains its organoleptic perspective when ingested.

The hard candy containing ginger extract contains active compounds, including gingerol. The gingerol may assist with nausea, dyspepsia, and motion sickness when ingested. The ginger extract may have at least a partial effect as an antagonist on 5HT-3 receptors in the brain that are implicated in nausea. When a person allows the hard candy to dissolve in their mouth, the active ingredients enter the bloodstream, and exert at least part of their mechanism on 5HT-3 receptors to provide relief for stomach upset and nausea.

The hard candy containing peppermint oil contains active substances, including menthol that, as part of their mechanism, may act via calcium channels to cause muscle relaxation. In addition, the active derivatives of peppermint oil may act on TRPM8 receptors in the colon to alleviate pain symptoms. The digestive symptoms of cramps, urgency, and bloating often share a common pathogenesis of aberrant digestive motility as an etiology. The immediate release of the active compounds from the peppermint oil allows for immediate relief of these complaints through direct relaxation effects on the muscles of the digestive tract and through interaction at TRPM8 receptors in the colon.

At step 112, the hard candy is administered. According to a method of administering the hard candy dose, when a person experiences digestive upsets, they may place the hard candy containing the botanical derivative into their mouth. The normal human digestive processes of the mouth, including mixing of the confection with saliva, may dissolve the confection and, with it, the active compounds. The blood flow through the buccal membranes may then provide for the rapid absorption of the active ingredients to enter the body and act at specific receptors that are implicit to the digestive complaints.

FIG. 2 is a graph showing the results of clinical trials using the hard candy dosages described herein. Specifically, drops from Example 1 and Example 2 were used. The x-axis shows time in days. The y-axis shows an expanded pain scale from 0 to 100, wherein 0 represents the worst pain and 100 represents little or no pain. Fourteen participants who regularly experienced gastrointestinal symptoms were surveyed at least once per week regarding their digestive symptoms. The gastrointestinal symptoms surveyed included urgency, bloating, diarrhea, and abdominal discomfort. The results of the survey were averaged and are shown on the graph in FIG. 2.

A baseline value was taken on day 0. A final survey was taken on day 21. The average of the patient surveys indicated improvements in their gastrointestinal symptoms. A statistical analysis of the data, the p-values, indicates that the improvements are statistically significant.

FIG. 3 is a bar graph showing improvements in common digestive symptoms when using the hard candy dosage described herein. The x-axis represents the results of a yes/no survey study. The y-axis represents the number of participants in the study. Each participant took at least 6 hard candy dosages per day, 3 ginger (Example 1) and 3 peppermint (Example 2). Of the 14 participants, 10 took the hard candy dosages at the onset of digestive symptoms which included abdominal discomfort, urgency, bloating, diarrhea, and nausea; the other 4 participants did not. Of the 10 participants who took the hard candy dosages at the onset of digestive symptoms, each participant reported that the hard candy dosages helped alleviate the symptoms within five minutes, as illustrated by the bar. This is considered an immediate clinical effect that can not be achieved through luminal digestive absorption. The test showed a p-value of 0.002 which is strongly statistically significant.

Exemplary embodiments of a hard candy dosage, a method of forming a hard candy dosage, and a method of administering a hard candy dosage are described above in detail. The ingredients and method steps illustrated are not limited to the specific embodiments described herein, but rather, the ingredients and method steps may be utilized independently and separately from other components described herein.

For example, the exact composition of the sugar base may be modified. Moreover, the method could be modified by adjusting the ingredients to provide for more rapid dissolution of the sugar base, thereby enabling more rapid release of the active compounds into the body.

Furthermore, different botanical derivatives with distinct anticipated health benefits could be used in the sugar base in addition to ginger and peppermint oil. Different products that utilize the same hard candy-based delivery system, but employing distinct botanical derivatives, may also be produced. Other botanicals include, but are not limited to cinnamon, fennel, turmeric, caraway, rosemary, lavender, cardamom, coriander, star anise, and lemongrass.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

What is claimed is:

1. A buccal delivery system for assisting with the relief of common digestive complaints, the system comprising:
a sugar base; and
at least one botanical derivative having at least one concentrated active compound infused into the sugar base;
wherein the active compound is present in an amount sufficient to provide relief from digestive distress;
wherein the at least one botanical derivative is not microencapsulated; and
wherein the at least one active compound is immediately released into systemic circulation through buccal absorption when administered

2. The delivery system of claim 1, wherein the system further comprises at least one natural flavoring.

3. The delivery system of claim 2, wherein the system is shaped into hard candy drop dosages

4. The delivery system of claim 3, wherein the hard candy dosages have favorable organoleptic properties.

5. The delivery system of claim 1, wherein the botanical derivative is infused into the carbohydrate base in a range of 0.1% to 5.0% by weight.

6. The delivery system of claim 1, wherein the active compound is infused into the carbohydrate base in a range of 0.01% to 5.0% by weight.

7. The delivery system of claim 3, wherein the active compound is infused into the carbohydrate base in an amount of from about 0.5 mg to 50 mg per hard candy drop.

8. A method of assisting with the relief of common digestive complaints comprising administering a buccal delivery system comprising a sugar base; and
at least one botanical derivative having at least one concentrated active compound infused into the sugar base;
wherein the active compound is present in an amount sufficient to provide relief from digestive distress;
wherein the at least one botanical derivative is not microencapsulated; and
wherein the at least one active compound is immediately released when administered.

9. The method of claim 8 further comprising at least one natural flavoring.

10. The method of claim 9 further comprising the system is shaped into hard candy drop dosages.

11. The method of claim 8 further comprising the hard candy dosages having favorable organoleptic properties.

12. The method of claim 8 further comprising administering a hard candy drop dosage containing a therapeutically effective amount botanical derivative in a range of 0.1% to 5.0% by weight.

13. The method of claim 8 further comprising administering a confection containing a therapeutically effective amount active compound in a range of from 0.01 to 5.0% by weight.

14. A method for preparing a buccal delivery system for assisting with the relief of common digestive complaints, the method comprising:
heating a sugar base to liquify;
infusing a botanical derivative into the sugar base to form a sugar mixture, wherein the botanical derivative contains at least one concentrated active compound;
wherein the botanical derivative is not encapsulated; and
cooling the sugar mixture into hard candy drop dosages;
wherein the dosage releases immediately.

15. The method of claim 14 further comprising infusing the sugar base with at least one additional natural flavoring.

16. The method of claim 14 further comprising infusing the sugar base with ginger extract containing about 10 wt. % gingerol.

17. The method of claim 14 further comprising providing a sugar base selected from the group consisting of cane syrup, brown rice syrup and corn syrup.

18. The method of claim 14 further comprising infusing the carbohydrate base with the botanical derivative in a range of 0.1% to 5.0% wt. %.

19. The method of claim 14 further comprising infusing the carbohydrate base with the active agent in a range of 0.01% to 5% weight.

20. The method of claim 14 further comprising hard candy dosages are in the range of from about 1.0 to 5.4 grams and contain from about 0.5 to 50 mg active compound.
